(19) United States
(12) Patent Application Publication
(10) Pub. No.: US 2026/0078026 A1
(43) Pub. Date: Mar. 19, 2026

(54) FOOD PROTEIN AND STARCH PURIFICATION AND WATER RECYCLING METHODS

(71) Applicant: ICM, Inc., Colwich, KS (US)

(72) Inventor: Charles C. Gallop, Gower, MO (US)

(21) Appl. No.: 19/108,067

(22) PCT Filed: Jun. 7, 2024
(86) PCT No.: PCT/US2024/033080

§ 371 Date: Feb. 28, 2025

Related U.S. Application Data
(60) Provisional application No. 63/472,251, filed on Jun. 9, 2023.

(51) Int. Cl. C02F 1/44; A23J 1/12; A23L 7/10; B01D 71/06; C02F 103/32; C07K 14/415

(52) U.S. Cl. CPC C02F 1/44; A23J 1/12; A23L 7/198; B01D 71/06; C07K 14/415; C02F 2103/32

(57) ABSTRACT

Various examples disclosed relate to methods for processing an agricultural commodity to produce protein and starch. The present disclosure includes methods that produce protein and starch in addition to recyclable water. The methods can leverage various filtration techniques to separate protein and starch from the recyclable water.

### CLAIM OF PRIORITY

This application claims the benefit of priority to U.S. Provisional Patent Application No. 63/472,251, filed on Jun. 9, 2023, which is incorporated by reference herein in its entirety.

### TECHNICAL FIELD

The invention relates generally to food processing technology. The invention relates more particularly, in various embodiments, to methods of solids separation in grain processing.

### BACKGROUND

Various types of grain are processed to isolate particular components therein, and often are processed in the context of a large volume of water. Additionally, grain processing can be done to separate out components such as starch and protein for various uses. For example, separated starch can be used in fermentation processes to produce ethanol. During such grain processing, a large amount water can be used.

### SUMMARY OF THE DISCLOSURE

In some aspects, the techniques described herein relate to a method of processing an agricultural commodity, the method including: separating a process stream in a production facility with a mechanical separation device into a liquid stream and a solids stream, wherein the process stream includes water and the agricultural commodity; clarifying a portion of the liquid stream with a filtration system to recover water from the liquid stream; isolating protein from the liquids stream; isolating starch from the solids stream; and conveying the recovered water to the process stream in the first production facility or to a second production facility.

In some aspects, the techniques described herein relate to a method including: receiving a process stream including a slurry of water and flour; separating the process stream into a liquid stream and a solids stream including protein and starch, wherein separating including flowing the process stream through a cross-flow filtration device; isolating the protein and starch from the solids stream; producing ethanol with the starch; and recycling the liquid stream.

In some aspects, the techniques described herein relate to a method of processing grain, the method including: receiving a process stream in a separation device, the process stream including a slurry of water and a processed agricultural commodity; separating the process stream into a liquid stream and a solids stream, with the separation device; filtering a portion of the liquid stream with a filtration system to recover water from the liquid stream; and recycling the water from the liquid stream to an upstream location.

In some aspects, the techniques described herein relate to a system for processing grain, the system including: a separation device for separating a slurry including the grain into a first solids stream and a first liquid stream; a feed tank for converting the first liquid stream to a second solids stream; and a filtration system for converting a second solids stream to a third solids stream and a permeate stream including water.

### BRIEF DESCRIPTION OF THE FIGURES

The drawings illustrate generally, by way of example, but not by way of limitation, various examples discussed in the present document.

FIG. 1 is a block diagram of a system for processing grain in an example.

FIG. 2 is a block diagram of a system for processing grain in an example.

FIG. 3 is a flow chart depicting a method of recycling water in a grain process system in an example.

FIG. 4 depicts the starch processing system in an example.

FIG. 5 depicts a starch filtration system in an example.

FIG. 6 depicts a starch filtration system in an example.

FIG. 7 depicts a starch filtration system in an example.

FIG. 8 depicts a starch filtration system in an example.

FIG. 9 depicts a starch filtration system in an example.

FIG. 10 depicts a starch filtration system in an example.

### DETAILED DESCRIPTION

Reference will now be made in detail to certain examples of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

A person knowledgeable in the below discussed applications would be able to understand how and where to leverage the invention to improve their process streams. The systems and layouts discussed herein can be of any configuration or design. For example, systems such as ethanol plants discussed herein can be configured as beverage grade facilities, fuel grade facilities, industrial type facilities, or of other appropriate configurations. Similarly, commodities processing facilities discussed herein can be of any design or configuration, such as for processing of various types of proteins, including but not limited to gluten, pea protein, whey protein, and others. The discussion herein pertains to processing a variety of different types of agricultural commodities, both plant and animal based. As such, the specific configurations described in this disclosure are in no way limiting.

Discussed herein are methods of processing starch and protein laden streams, such as wheat, using less water while allowing purity of coproducts produced during processing. The method leverages the use of cross-flow filtration to reduce the overall amount of incoming water needed, as well as improve the quality of the resulting concentrated solid and liquid streams. For example, when wheat is processed, it may be desirable to separate starch from protein (e.g., gluten). In such cases, starch can be sent to produce other byproducts like ethanol via fermentation. The protein can be used in other ways. The methods herein also allow for production of protein with good purity and good protein content.

A variety of different plant products can be processed through the processes and system discussed herein. For example, in some cases, cereals such as wheat, oat, rice, and corn, can be used. In some cases, starch crops such as yellow pea, chickpea, fava bean, mung bean, and potato can be used. In some cases, oilseeds, such as soybean, sunflower, rapeseed, canola, or lupin can be used. In some cases, aquatic plants such as kelp, seaweed, dulse, or sea vegetables can be used. Additional or other suitable types of vegetation can be processed in the systems discussed herein.

Previous methods would discharge process water to a waste water treatment facility. This would prevent recycling or reuse of water, and prevent production of increased protein purity. Moreover, some previous methods would evaporate a portion of water during processing, causing excessive energy consumption. Some such processes also relied on chemical additions to harden distilled water used in protein formation.

By comparison, the discussed methods have several advantages, some of which are unexpected. First, the water used in the process can be treated so that it is food grade water, and recycled or reused, sent back to earlier parts of the process. This can help reduce the need for fresh water in the process, such as by up to eighty percent. The recycling of water can help in efficiency and cost reduction in the overall process.

Meanwhile, the starch separated out can be of higher quality for fermentation and ethanol production. Without a forced flow of new water to the processes, there is less overall waste and exiting of fluid from the system. This helps produce more concentrated starch for fermentation and ethanol production.

Additionally, the protein (e.g., gluten) produced can be produced more efficiently and have better protein content, quality, or both. For example, protein purity produced by the methods discussed herein can go up by about ten percentage points, correlated to the ion content of recycled water. This can occur, for example, with gluten production in wheat processing, because of calcium ion content in the recycled water. This can help create gluten with better elasticity, in a more efficient manner.

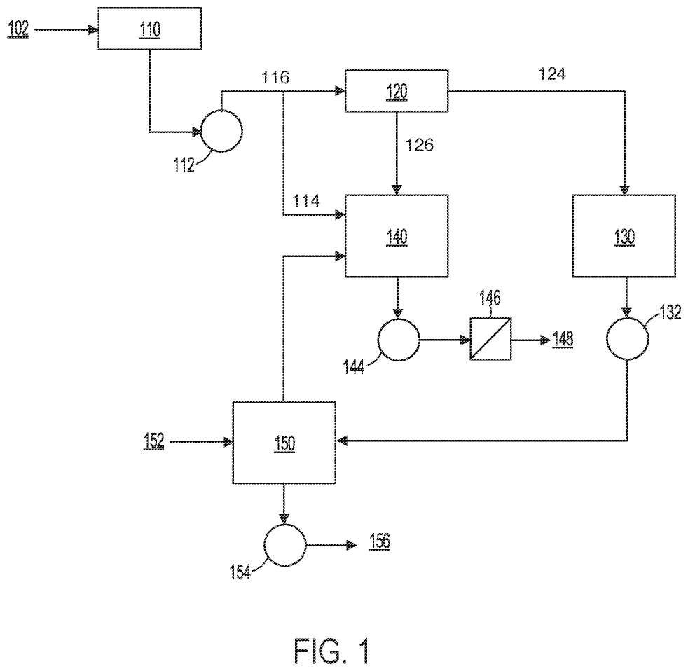

FIG. 1 depicts an example wheat starch processing system 100, in which water can be recycled for use in various food grade processing systems. The System 100 in FIG. 1 can include feed tank 110, separators 120, feed tank 130, retentate tank 140, and separation system 150. The system 100 can be used to separate out components of a process stream including a slurry.

For example, a starch slurry 102 can be processed in the wheat starch processing system 100. The starch slurry 102 can be from a wheat starch plant, such as is partially processed, and arrives at the wheat starch processing system 100 in a slurry state ready for processing to produce both an ethanol slurry 148 and a water permeate stream 156. The starch slurry 102 can be a slurry of water and a processed agricultural commodity, such as a milled flour. For example, rice, wheat, corn, or other flour, or other milled plant product, can be in a slurry form.

The incoming starch slurry 102 can be flowed into the feed tank 110. The feed tank 110 can maintain a temperature between about 60° C. to about 100° C., such as to cause the starch to gelatinize and a residence time of about 30 to about 60 minutes to convert insoluble starch in the slurry to soluble starch, with or without the use of enzymes. The slurry may have suspended solids content of about 26% to about 50%, which includes starch, fiber, protein, and oil. Other components in the feed tank 110 may include, grit, salts, and the like, as is commonly present on raw incoming grain from agricultural production, as well as recycled waters that contain acids, bases, salts, yeast, and enzymes. The pH of the slurry can be adjusted for associated enzyme performance. In one example, the pH of the slurry can be adjusted to about 4.5 to 6.0 (depending on enzyme type) in the feed tank 110.

The starch slurry 102 can be separated into two process streams at a separation device 112. The separation device 112 can be, for example, a mechanical separation device, such as a centrifuge, a decanter, or any other type of separation device. Any number of separation devices may be used, ranging from one, two, three, or more separation devices. For example, a plurality of filters in series may be used. In another example, a size exclusion device may be used. In some cases, cross-flow filtration can be used.

Coming out of the separation device 112, the starch slurry 102 can be separated into a filtration system bypass stream 114 and screener feed stream 116. The filtration system bypass stream 114 can include starch and solids, and be flowed into the retentate tank 140.

The screener feed stream 116 can include additional starches and solids to be filtered out, and be flowed into the separators 120 to further separate out those solids and starches. The separators 120 can leverage gravity separation to further separate out solids and liquids in the stream. The separators 120 can be, may include, but are not limited to, a dissolved air floatation device, an open tank dissolved air floatation device, a filter press, a DSM screen, a centrifuge, a vibrating screener a sieve bend screen, and the like. Multiple separators can be used. At the separators 120, the screener feed stream 116 can be separated into screener solids stream 126 and filter feed stream 124. The streams can be gravity fed 122.

The screener solids stream 126 can be flowed to the retentate tank 140. The screener solids stream 126 can include the filtered-out solids (e.g., the retentate after flow through the separators 120), such as including starches. The retentate stream can be flowed through an additional separator 144 and optionally a heat exchanger 146 to help thermally regulate the stream. The retentate stream can then be flowed out of the wheat starch processing system 100 to an ethanol slurry 148, for processing at an ethanol plant.

The filter feed stream 124 can be flowed into the feed tank 130. The feed tank 130 can be, for example, similar to the feed tank 110. The filter feed stream 124 can be flowed out of the feed tank 130 through another separator 132 as desired to the separation system 150.

The separation system 150 can take in starch in process streams from the feed tank 130, in addition to other incoming starch-containing process streams. For example, the separation system 150 can receive the filter feed stream 124 through separator 132 and a second process stream 152. The separation system 150 can be used to separate out starches or other solids from water in the process stream, resulting in a filter retentate stream 142 containing starch and other solids, which can be cycled back to the retentate tank 140, and a water permeate stream 156, which is chiefly water to be recycled.

The water permeate stream 156 can be recycled upstream. For example, the water can be used in various tanks or slurries as desired. Water recycled from other portions of the system can also be recovered and recycled.

The separation system 150 can include one or more filtration devices for separating the starch and the water within the incoming streams. For example, the separation system 150 can include a centrifuge device. Such a device may include, but is not limited to, a sedicanter, a decanter centrifuge, a disk stack centrifuge, a cyclone, a hydrocyclone, a settling tank, filtration devices, and the like, or combinations thereof.

In some cases, such a centrifuge can be followed by a membrane filtration device. In an example, an open channel membrane device can be used. For example, a membrane filtration device with discrete retentate channels in a module can be used. Inlets and outlets can be diagonally opposed depending on the desired flow pattern. The membrane filtration device can offer improved fluid dynamics providing true linear scalability, optimized yields, decreased downstream process time, and reduced costs for an efficient separation. The membrane filtration device can include a broad range of membrane materials, channel height, membrane area, and processing parameters. Other factors may also include, but are not limited to, processing temperature, membrane pore sizes, different types of materials, and different types of solid polymer materials (i.e., face area).

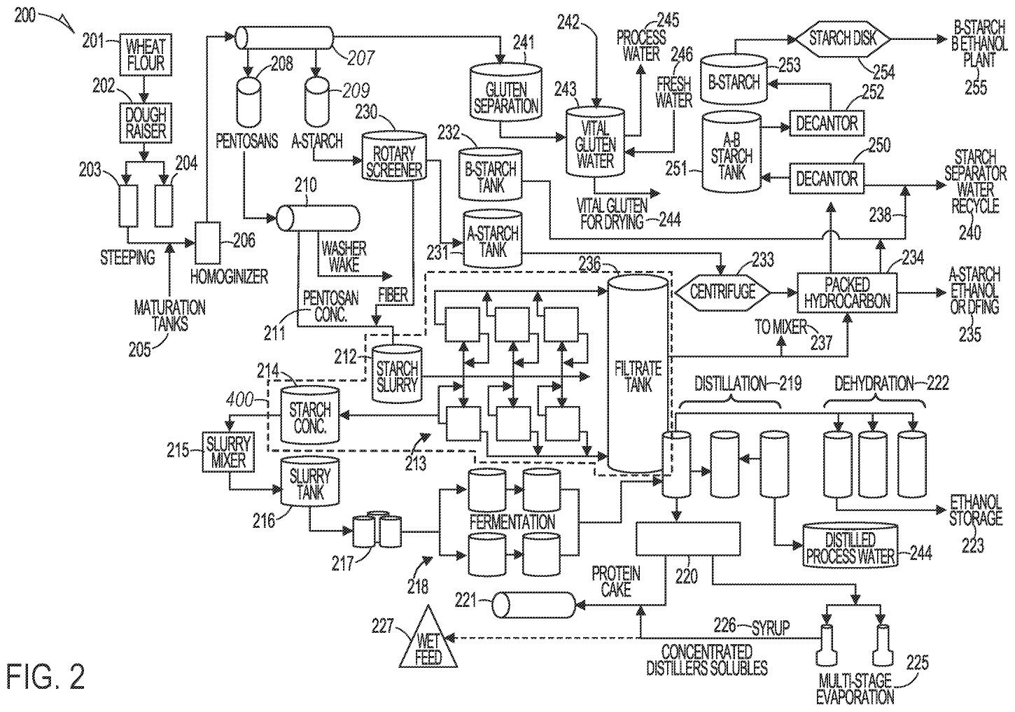

In some cases, the separation system 150 can further include one or more other separation devices to help filter out the starch in the process stream, such as a stacked disk centrifuge, a packed hydroclone, or other component. The outgoing filter retentate stream 142 can be sent to an ethanol plant via the retentate tank 140. The water permeate stream 156, by comparison, which can include chiefly water, can be recycled to other parts of this system 100, or other linked systems, such as those discussed below with reference to FIG. 2.

The water permeate stream 156 can be upwards of 90% to 99% water, such as about 90, 91, 92, 93, 94, 95, 96, 97, 98, or 99% water. The water permeate stream 156 can optionally be sent to other portions of the system 100 where incoming water is desired.

FIG. 2 depicts a larger grain processing system 200 that blends a wheat processing method and an ethanol processing method with cross flow filtration in-between. The cross-flow filtration allows for recycling of water within the overall system. The system 200 can include a variant of the system 100 for processing of a starch slurry discussed above.

In an example method of using the system of FIG. 2, a food wheat liquid stream can be intercepted. The stream can be separated into solids and liquids, with the liquids including water. Specifically, the methods discussed herein can allow for separation of suspended solids from liquid and allow for recycling to the front end of the protein separation step, and concentrate the suspended solids. In the system 200, water can be recycled. In an example, solids can be removed from the process stream, such as by reverse osmosis, through a variety of filtration steps, to produce a recyclable water stream. In an example, micro filtration or other filtration can be used. Meanwhile, the starches, such as particular sugars, can be captured for hydrolyzing and isolation.

In the system of FIG. 2, wheat flour can be received and processed, producing a process stream such as a slurry including wheat flour and water. Such a process stream can be separated to produce suspended solids separate from water. The separation methods can remove both suspended and dissolved solids from the process stream.

The separation methods can use size exclusion. Size exclusion can be performed, for example, through screening or filtration. Another methodology of discharge water processing may be a form of anaerobic or aerobic digestion, such as to produce renewable methane.

In an example, the separation methods can include using a plate and frame apparatus for cross-flow filtration of the process stream and separation of solids and liquids. Such an apparatus can include parallel plates with a predetermined gap, where water flows through the gaps. The plates can include a membrane, such as a polymeric membrane, which a desired porosity to capture suspended and dissolved solids from the process stream. In some cases, the membrane on the plates can be hydrophilic. In some cases, the membrane on the plates can be hydrophobic. The process stream can be pushed through the apparatus, and the water will continue to flow through, while solids will be taken up by the membrane on the plates. Continuous filtration can be done when the system is in used. For example, a constant feed of the process stream can be pushed through this apparatus. A portion of the recycled flow can be bled out with filtrate as desired.

The separation methods can be done in multiple steps. In an example, fractionation of suspended solids based on size can be done to allow for purification of each solids component. For example, a first stage of filtration may remove fibrous substrates, such as wheat midds or bran from the rest of the process stream. The next processing step may have a less porous filter to remove insoluble starch from the rest of the solution. The next separation step could remove fine suspended solids and some dissolved solids such as pentosans, single cell organisms, and organic acids or salts. The final separation step could remove the remaining dissolved solids such as a reversed osmosis process. This stepwise approach allows for complete solid and liquid separation and purification of each component.

In the example system shown in FIG. 2, several separation apparatuses are shown, so that starch and protein can be separated from the water, and isolated with a desired purity. The water, once separated from the solids, can be cycled back, such as to create the initial wheat slurry, or for other purposes in the system of FIG. 2.

The ability to recycle food grade water back to the food grade facility may reduce the amount of fresh water intake to produce the food grade products and reduces the amount of energy required to process the water before discharge. In some cases, this discharged water may go to a secondary processing facility, such as an ethanol plant to convert the starch that has been separated from the protein to secondary coproducts such as ethanol.

The methods discussed herein have increased separation efficiency and protein purification of wheat gluten through the water reuse/recycle methodology by way of recycling soluble minerals that are in the recycled water. Such recycled water does not need additional chemical or additives to maintain appropriate ion levels for use in grain processing or ethanol production.

In the example system 200, incoming wheat flour 201 can be run through a dough raiser 202. The processed wheat starch can be hydrated through the addition of water at the tanks 203, 204. At the tanks 203, 204, the wheat dough water mixture can be steeped. As desired, additional mixture from the maturation tanks 205 can be used or mixed with the flow from the tanks 203, 204. The mixture can be moved to a homogenizer 206 to process a homogenous wheat starch slurry.

The slurry can be separated in a tricanter 207, a type of centrifuge apparatus. Here, three streams can be produced: one stream of pentosans to tank 208, one stream of A-starch to tank 209, and one stream of protein to gluten separation apparatus 241. The A-starch slurry can include six-chain sugars, while the pentosans are five-chain sugars.

The pentosans slurry from the tank 208 can be flowed to decanter 210. Here, water can be separated out as washer wake, and potentially recycled elsewhere. Meanwhile, pentosan concentrate 211 can be flowed into the starch slurry tank 212 for further separation of starch from water. When isolated, the pentosans in the pentosans slurry can be used in anaerobic digesters.

The A-starch slurry from the tank 209 can be flowed through a separation device, such as a rotary screener 230. The rotary screener 230 can produce a stream of fiber, which can be directed to the starch slurry tank 212, in addition to an A-starch stream, which can be delivered to A-starch tank 231. The A-starch stream can be separated out through a centrifuge 233 and a packed hydroclone 234. The packed hydroclone 234 can produce several streams: a starch stream 235 and a water stream 238. The starch stream 235 can be sent to an ethanol plant for processing. The water stream 238 can be sent to a decanter and/or sent to a recycling stream, so that the water can be recycled back into the system 200 or other process systems as desired.

In an example, the water stream can be cycled through another decanter 250 for used in another A-starch tank 251. Such a water stream could be further recycled coming out of the A-starch tank 251 and processed by another decanter 252. In that example, the water stream can be recycled through a B-starch tank 253. A stream containing B-starch from the B-starch tank 253 can be processed by a stacked disc 254, such as a separation device, to produce a B-starch stream 255 for use in an ethanol plant.

Meanwhile, the stream received in the gluten separation apparatus 241 can be subject to separation of protein. The gluten-containing stream from the starch concentrate tank 214 can be mixed in a vital gluten water tank 243. The vital gluten water tank 243 can receive fresh water 246 in addition to the gluten containing stream from the gluten separation apparatus 241 and optionally an additional process stream 242. At the vital gluten water tank 243, vital gluten 244 can be separated out for drying, as can process water 245. Optionally, process water 245 can be further filtered and recycled back into the system 200 where desired.

The starch slurry tank 212 can receive the pentosan concentrate 211 and fiber from the rotary screener 230. The starch slurry tank 212 can produce a stream of starch that is separated out to a starch stream and a recycled water stream, similar to the separation system 150 discussed above. The starch stream from the starch slurry tank 212 can be run through several membrane filtration devices 213. The several membrane filtration devices 213 can be similar to those described above with reference to separation system 150.

The resulting water-containing filtrate can be sent to the filtrate tank 236, while the starch concentrate can be sent to starch concentrate tank 214. The water-containing filtrate from the filtrate tank 236 can optionally be sent to a mixer 237 for use elsewhere. The water-containing filtrate from the filtrate tank 236 can optionally be sent to the packed hydroclone 234 to help process the A-starch stream from the A-starch tank 231 being sent to the starch stream 235.

The starch concentrate at the starch concentrate tank 214 can be mixed into a slurry at the slurry mixer 215, such as with the addition of water, which may be recycled water from elsewhere in the system 200. The wheat slurry can be sent to a slurry tank 216 and then to tanks 217 on the way to being processed for ethanol.

The starch slurry can be fermented at the fermentation tanks 218, then distilled and dehydrated at distillation 219 and dehydration 222, respectively. The resulting ethanol can be sent to ethanol storage 223. Distilled process water can be flowed out of the distillation 219 to the distilled process water tank 224. A portion of the starch slurry can run through processor 220, and be separated into solid protein cake stored at location 221, and a stream run through multi-stage evaporators 225. The multi-stage evaporators 225 can produce a syrup 226 of concentrated distiller solubles, which can be used to produce wet feed 227.

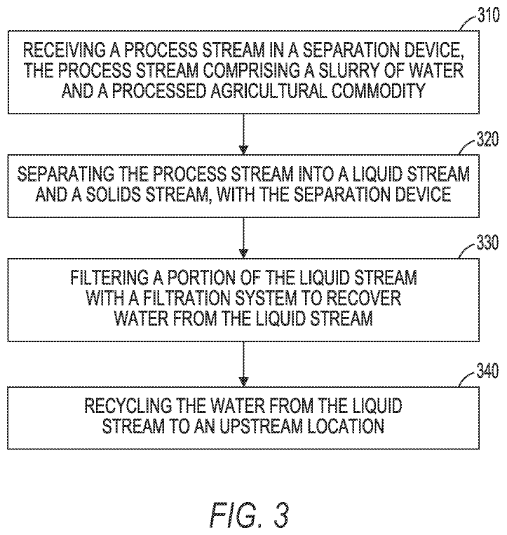

FIG. 3 depicts a flowchart of an example method for processing grain.

The method can include at step 310 receiving a process stream in a separation device, the process stream comprising a slurry of water and a processed agricultural commodity. Here, the processed agricultural commodity can be a grain that has been milled into a flour and mixed into a slurry. For example, the grain can be wheat, corn, barley, or rice. The grain can be milled to produce a flour. The flour can be put through a dough riser, and then put in tanks with water to produce a slurry. The mixture can be homogenized prior to further processing.

At step 320, the method can include separating the process stream into a liquid stream and a solids stream, with a separation device. The separation device can be, for example, a mechanical separation device, such as a centrifuge or a decanter, as desired. In some cases, other technologies, such as paddle screen, Rotary press, multi-zone separation apparatuses, mechanical sieves, filter press, and combinations thereof can be used.

At step 330, the method can include filtering a portion of the liquid stream with a filtration system to recover water from the liquid stream. The filtration system can include one or more filtration devices. The filtration devices can include, for example, a size exclusion device. In an example, the filtration device can include a membrane-based device, such as a cross-flow filtration device. The filtration devices can produce a water stream and a starch stream.

At step 340, the method can include recycling the water from the liquid stream to an upstream location. The water stream can be routed from the filtration device to other parts of the method, such as for use in producing a slurry or in a tank. In some cases, other technologies, such as paddle screen, Rotary press, multi-zone separation apparatuses, mechanical sieves, filter press, and combinations thereof can be used. In some cases, the water can be recycled outside of the particular system to another system, such as a food facility or an ethanol facility. In some cases, other technologies, such as paddle screen, Rotary press, multi-zone separation apparatuses, mechanical sieves, filter press, and combinations thereof can be used.

Optionally, the method 300 can include isolating protein (e.g., gluten) from the liquids stream, isolating starch (e.g., A-starch, B-starch, or pentosans) from the solids stream, producing ethanol with the starch, or combinations thereof. In any of these additional isolation processes recycled water can be used.

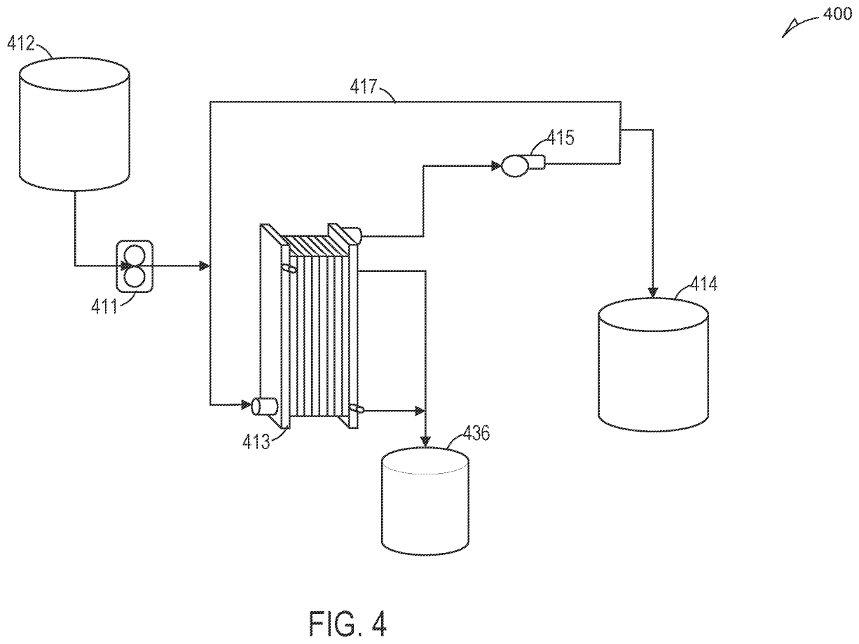

FIGS. 4 to 10 are schematic diagrams depicting example variations on the starch processing system 400 depicted in FIG. 2. Generally, such as shown in the example of FIG. 4, such a starch processing system 400 can include a starch source 412, a filter feed pump 411, one or more membrane separation devices 413, a filter recirculation pump 415, a retentate tank 414, and a filtrate tank 436. The additional variants on the processing system, such as those shown in FIGS. 4 to 10, can including differing numbers of membrane devices, parallel or series set-ups, optional secondary filtration or tertiary filtration, and other variations. FIG. 4 depicts a general example of such a system 400 with a single membrane filter device 413.

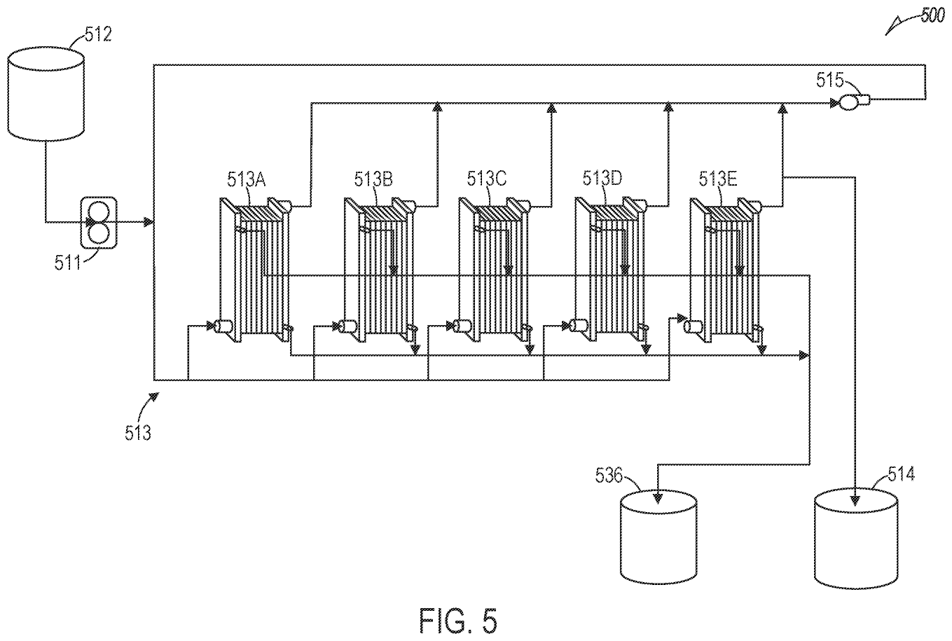

FIG. 5 depicts a starch processing system 500. The starch processing system 500 can be a loop configuration including the filter recirculation pump 515. Here, the starch slurry can enter from the starch source 512, and be moved through the starch processing system 500 by action of the filter feed pump 511. The slurry can be filtered through membranes 513, such as including multiple membrane devices 513a, 513b, 513c, 513d, 513e, in parallel. In other examples, multiple membrane devices can be used in series. At the membranes 513, a portion of the stream can be retained on the surface of the membranes and prevented from flowing through.

A portion of the stream can be separated out in the filtrate tank 536, a portion can be filtered into the retentate tank 514, and a portion can be recirculated through the filter recirculation pump 515. Similar to the components described above with reference to FIG. 2, the filtrate tank 536 can receive a portion of the stream that is water-containing filtrate, while the retentate tank 514 can receive a portion of the stream that is a concentrated starch.

The flow rate and pressure of the starch processing system 500 can be regulated by the filter feed pump 511 and the filter recirculation pump 515. The system starch processing system 500 pumps can act as a control loop for the recirculation pressure. In other examples, additional amounts and types of pumps can be used according to the amount and type of membrane devices.

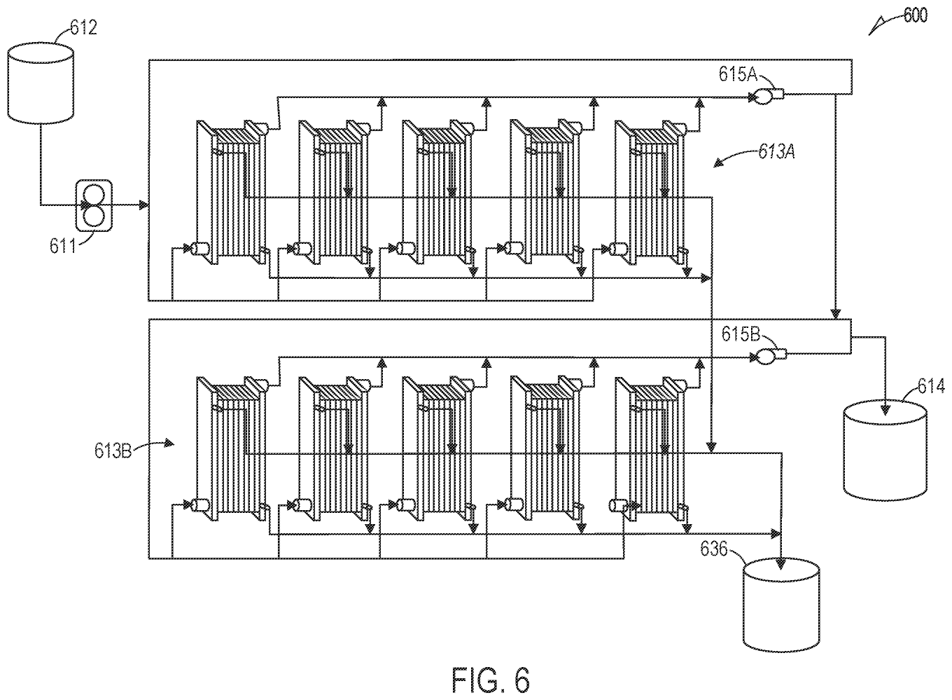

FIG. 6 depicts a starch filtration system 600. The system 600 can include starch source 612, filter feed pump 611, first membrane device bank 613a, second membrane device bank 613b, first filter recirculation pump 615a, second filter recirculation pump 615b, retentate tank 614, and filtrate tank 636. Here, the starch slurry comes into the system and is run through first membrane device bank 613a and second membrane device bank 613b in parallel. Each of the first membrane device bank 613a and the second membrane device bank 613b is regulated by its own first filter recirculation pump, 615a or 615b respectively.

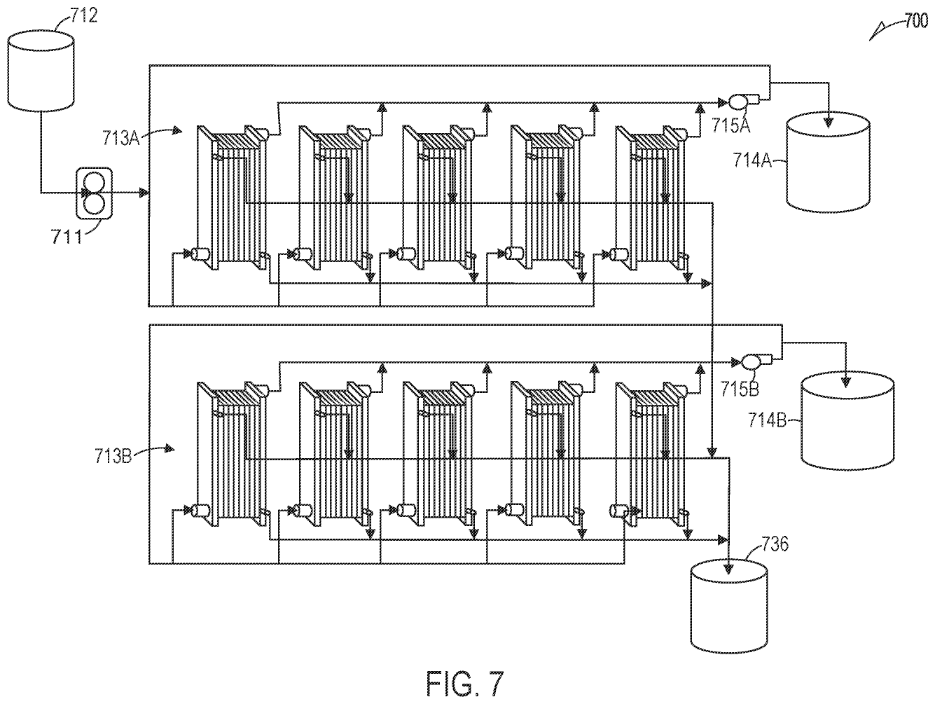

FIG. 7 depicts a starch filtration system 700 with two different types of filtration, one in the primary filtration membrane device bank 713a, and the other in the secondary filtration membrane device bank 713b. Here, the starch filtration system 700 can include the filter feed tank 712 and filter feed pump 711. The primary filtration membrane device bank 713a can be associated with its own filter recirculation pump 715a and retentate tank 714a. These tanks can be used to adjust the stream qualities such as pH, temperature, such as with addition of enzymes, emulsifiers, flocculants, or others. The secondary filtration membrane device bank 713b can be associated with its own filter recirculation pump 715b and secondary retentate tank 714b. A single filtrate tank 736 can be used.

Here, the stream including the starch slurry can flow from the filter feed tank 712 through the filter feed pump 711 to the primary filtration membrane device bank 713a first. The primary filtration membrane device bank 713a can be, for example, a bank of membrane filtration devices, such as ultra-filtration devices. The recirculation pump 715a can regulated the flow in this bank. A portion of the filtered stream can be flowed to the retentate tank 714a. A secondary portion of the filtered stream can be flowed to the secondary filtration membrane device bank 713b.

The secondary filtration membrane device bank 713b can be in series with the primary filtration membrane device bank 713a. The secondary filtration membrane device bank 713b can have its own filter recirculation pump 715b. In this example, the secondary filtration membrane device bank 713b can include a type of filtration devices different from the first bank, such as reverse osmosis filtration devices. The stream filtered through the secondary filtration membrane device bank 713b can be split into a secondary retentate tank 714b and the single filtrate tank 736.

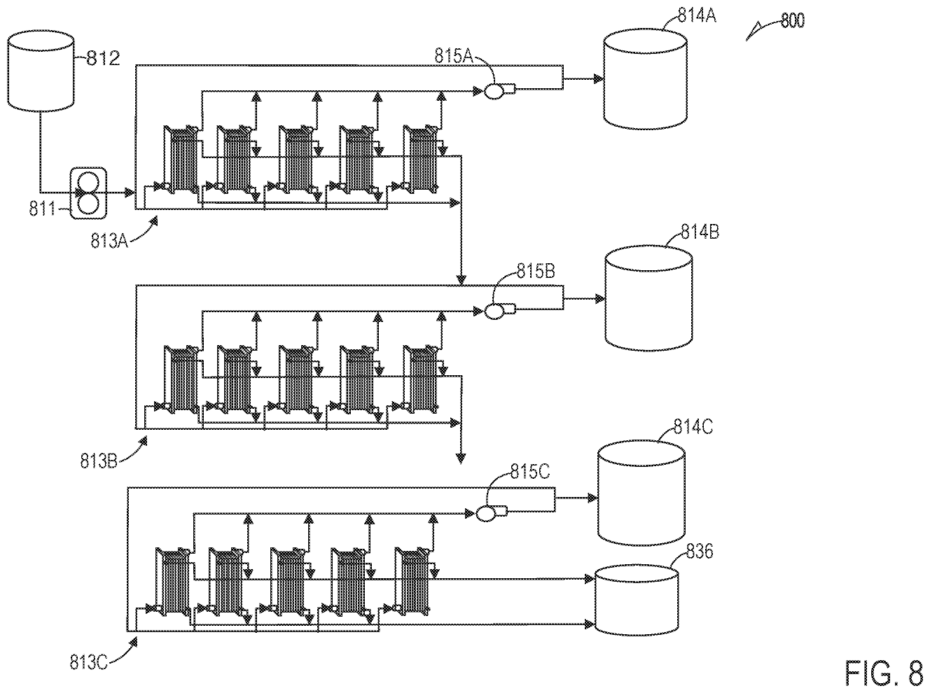

FIG. 8 depicts a starch filtration system 800 with three different types of filtration in series. The starch filtration system 800 can include filter feed tank 812; filter feed pump 811; first bank of membrane devices 813a with first filter recirculation pump 815a and first retentate tank 814a, second bank of membrane devices 813b with second recirculation pump 815b and second retentate tank 814b; third bank of membrane devices 813c with third recirculation pump 815c and third retentate tank 814c; and filtrate tank 836.

In the starch filtration system 800, the first, second, and third banks of membrane devices can be three different types of membrane filtration, such as ultra-filtration, nano-filtration, and reverse osmosis. The first, second, and third banks of membrane devices here can be in series, such that a starch slurry from the filter feed tank 812 can flow through them in sequence, being further filtered and separated along the course of the starch filtration system 800.

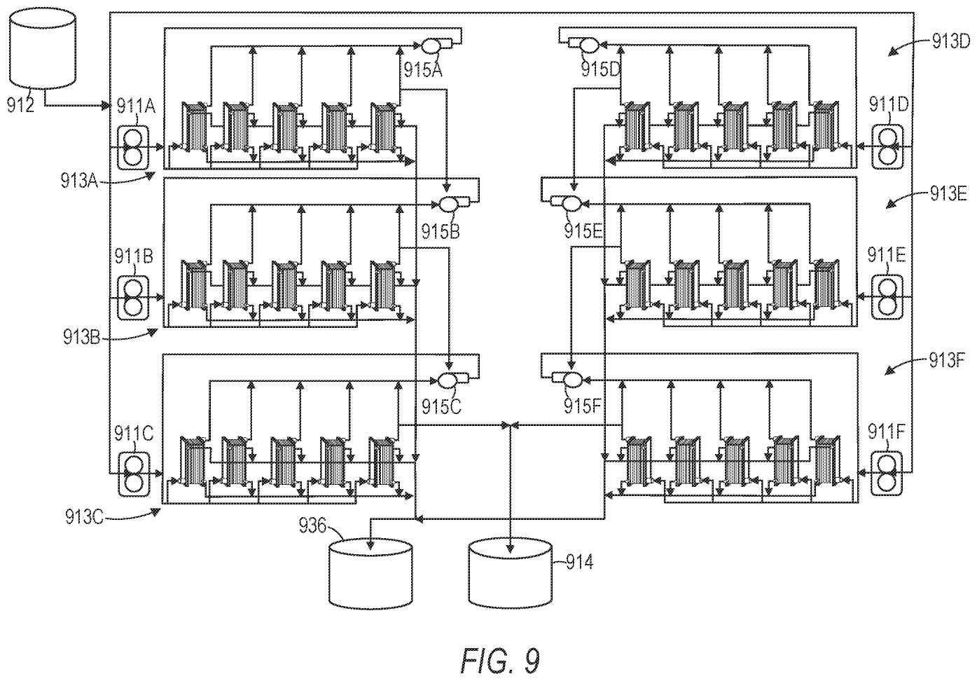

FIG. 9 depicts example starch filtration system 900 with multiple banks of membrane devices. The system 900 can include filter feed pumps 911a, 911b, 911c, 911d, 911e, and 911f; starch source 912; membrane devices 913a, 913b, 913c, 913d, 913e, and 913f; retentate tank 914; filter recirculation pumps 915a, 915b, 915c, 915d, 915e, and 915f; and filtrate tank 936.

The different banks a to f in starch filtration system 900 can allow for filtration of a large volume of starch slurry simultaneously. For example, each of the banks can be run in parallel with the others. The control loops for each bank, with their respective pumps, can be run independently of each other. As needed, such as for cleaning, one or more of the banks can be deactivated while the other banks continue to filter the stream. The various banks can have a common output at the retentate tank 914 and filtrate tank 936 for collection of retentate and filtrate, respectively.

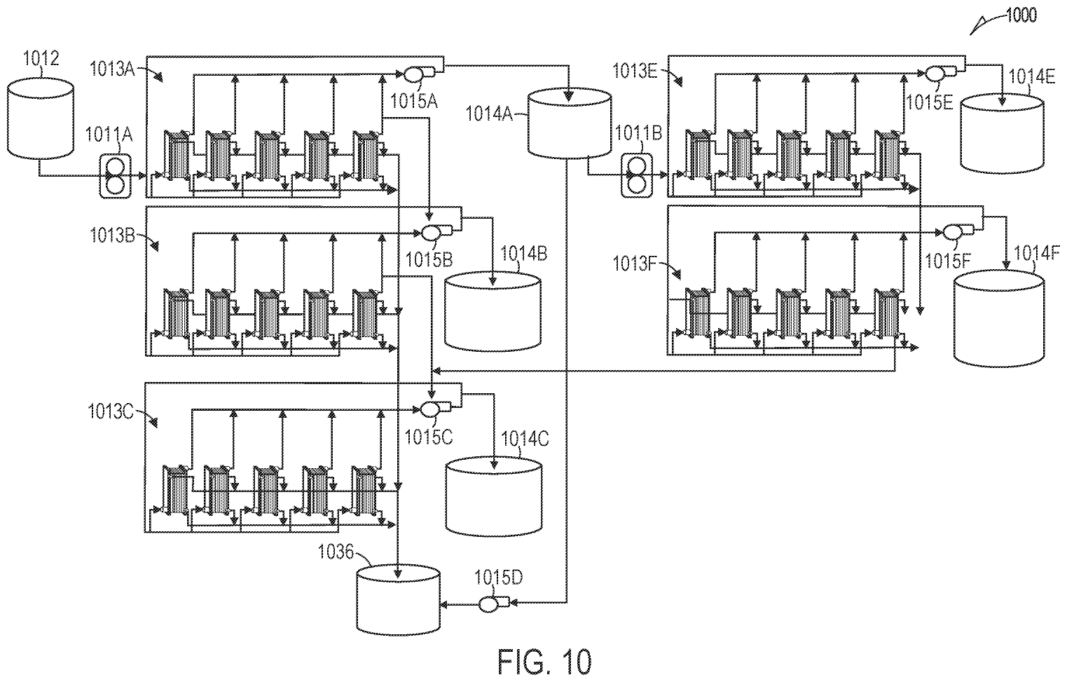

FIG. 10 depicts a starch filtration system 1000. The system 1000 includes starch slurry tank 1012. Here, five banks of membrane filtration devices are shown, separated into two lines that run in parallel each other.

The first bank can include first filter feed pump 1011a; first membrane filter device bank 1013a, first filter recirculation pump 1015a and first retentate tank 1014a; second membrane filter device 1013b with second filter recirculation pump 1015b and second retentate tank 1014b; third membrane filter device 1013c with third filter recirculation pump 1015c, third retentate tank 1014c; filtrate tank 1036; and first bank recirculation pump 1015.

The second bank of membrane filter can include fourth membrane filter device bank 1013e with fourth filter recirculation pump 1015e and fourth retentate tank 1014e; and fifth membrane filter device bank 1013f with fifth filter recirculation pump 1015f and fifth retentate tank 1014f.

Here, the starch slurry can run through a first filtration at the first membrane filter device bank 1013a, such as an ultra-filtration, with a portion of the stream going to the first retentate tank 1014a. The stream can then run through a second filtration at the second membrane filter device 1013b, such as nano-filtration, with a portion of the stream going to the second retentate tank 1014b. The stream can then run through a third filtration at the third membrane filter device 1013c, such as reverse osmosis, with a portion of the stream going to the third retentate tank 1014c.

The remaining stream can flow to the filtrate tank 1036. This stream can be recirculated via the recirculation pump 1015d to the first retentate tank 1014a. From the first retentate tank 1014a, a portion of the process stream can be sent to the second bank of membrane based filtration devices.

This portion of the stream can be further filtered at the fourth membrane filter device bank 1013e, and then the fifth membrane filter device bank 1013f in series. Portions of the stream can be separated out into the fourth retentate tank 1014e and fifth retentate tank 1014f, respectively. The fourth membrane filter device bank 1013e can, for example, be an ultra-filtration device. The fifth retentate tank 1014f can be, for example, a nano-filtration device. A portion of the stream from the fifth retentate tank 1014f can be cycled back to the first bank of membrane filtration devices.

### EXAMPLE

Various examples of the present disclosure can be better understood by reference to the following Examples which are offered by way of illustration. The present disclosure is not limited to the Examples given herein.

### Example 1. Starch Slurry Stream Processing

An example batch of a starch slurry stream was processed in a system similar to that shown and discussed with reference to FIG. 1 above. The starch slurry stream was received from a wheat starch plant, and run through the system 100. Here, the starch slurry was run through a feed tank, and split into a screener feed stream and a filtration system bypass stream. The filtrations bypass stream was run to a retentate tank. The retentate tank produced an ethanol slurry blender stream.

The screener feed stream was run to a separator. The separator produced a filter feed and a screener solids feed. The filter feed stream was run to a feed tank, which was in turn run to a filtration system. The filtration system is a cross-flow filtration with polymeric membranes. The filtration system received the stream from the feed tank, in addition to an incoming second process stream, and was used to separate the water out of the stream to produce the permeate to wheat starch plant stream, and a separate filter retentate stream going back to the retentate tank.

The permeate to wheat starch plant stream, separated out from the starch, was produced with less than 3% starch therein. The permeate to wheat starch plant stream was recyclable water for use in another system, or elsewhere in this system.

A summary of the properties of each portion of the process stream during the method, including gpm, density, flow rate, total starch, and water in the process stream at each point in the system is shown below in Table 1:

TABLE 1
Example process flow.

| | | Glutinous | | | | | |
|---|---|---|---|---|---|---|---|
| | | proso | | | | | |
| | | millet | | | Total | Total | |
| | FIG. | starch | Density | Flow rate | starch | starch | Water |
| Stream | 1 | (gpm) | (SG) | (lb./hr.) | (%) | (lb./hr.) | (lb./hr.) |
| Starch | 102 | 275 | 1.12 | 154,123.2 | 29% | 44,465.7 | 109,427.5 |
| slurry from | | | | | | | |
| wheat starch | | | | | | | |
| plant | | | | | | | |
| Screener | 116 | 210 | 1.12 | 117,694.1 | 29% | 34,131.3 | 83,562.8 |
| feed | | | | | | | |
| Screener | 126 | — | 1.11 | 6,880.5 | 34% | 2,327.8 | 4,552.7 |
| solids | | | | | | | |
| Filter feed | 124 | 198 | 1.12 | 110,813.6 | 29% | 31,803.5 | 79,010.1 |
| Filtration | 114 | 65 | 1.12 | 36,429.1 | 29% | 10,564.4 | 25,864.7 |
| system | | | | | | | |
| bypass | | | | | | | |
| Filter | 142 | 120 | 1.13 | 67,854.2 | 45% | 30,643.6 | 37,210.6 |
| retentate | | | | | | | |
| Ethanol | 148 | 197 | 1.13 | 111,163.9 | 39% | 43,535.8 | 67,628.0 |
| slurry | | | | | | | |
| blender | | | | | | | |
| Permeate to | 156 | 85 | 1.01 | 42,959.3 | 3% | 1,159.9 | 41,799.4 |
| wheat starch | | | | | | | |
| plant | | | | | | | |

The separation of water and starches was successfully done to produce the permeate to wheat starch plant stream, which could be successfully recycled. Here, the “Water” column of the Table 1 exhibits the movement of water through the system. Optionally, the method of the Example can include isolating protein (e.g., gluten) from the liquids stream, isolating starch (e.g., A-starch, B-starch, or pentosans) from the solids stream, producing ethanol with the starch, or combinations thereof. In any of these additional isolation processes recycled water can be used, such as water pulled from the system at different points.

### Example 2. Wheat Flour Processing

Wheat flour was processed in an example system to determine relevant flour characteristics and an estimation of the processing results. Ten samples were submitted for laboratory testing. The samples were “Gluten E” produced through a processing method similar to that shown in FIG. 2 above. The Gluten E sample was analyzed in a variety of methods, including the analysis methods summarized in Table 2:

TABLE 2
Analysis methods.

| Analysis | Test Method |
|---|---|
| Moisture | ICC 100/1, Flottweg WI-0041 |
| Ash content (ds/ds) | ICC 104/1, Flottweg WI-0042 |
| Soluble solids (ds/ds) | A.O.C.S. Official Method Ba 11-65, |
| | Flottweg WI-0056 |
| Soluble protein (NSI) | A.O.C.S. Official Method Ba 11-65, |
| | Flottweg WI-0056 |
| Fat (based on ds) | VDLUFA Method book III, 5.1.1 B, |
| | Flottweg WI-0058 |
| Stirring test | Flottweg WI-0060 |
| Gluten index | ICC 155, Flottweg WI-0061 |
| Wet gluten content | ICC 155 / ICC 137/1, Flottweg WI-0061 |
| Protein content (ds/ds) | ICC 167, Flottweg WI-0063 |
| Glutopeak | Flottweg WI-0064 |
| Sieve analysis | ICC 207, Flottweg WI-0065 |
| Particle size distribution | ISO 13320: 2020, Flottweg WI-0068 |
| Falling number | ICC 107 |
| Farinograph | ICC 115/1 |
| Sedimentation value | ICC 116/1 |
| Starch content | ICC 122/1 |
| Amylograph | ICC 126/1 |
| “Abstehnote” | Method Berliner |

Based on the above test methods, the initial wheat flour performed as summarized below in Table 3, while the produced gluten performed as summarized in below Table 4, and the produced starch performed as summarized in below Table 5:

TABLE 3
Testing results for wheat flour prior to processing.

| Property | Target Range | Measured Property |
|---|---|---|
| Moisture (% w/w) | <13.5 | 12.1 |
| Ash content ds/ds (% w/w) | <0.8 | 0.74 |
| Fat based on ds (% w/w) | <1.5 | 0.4 |
| Soluble solids ds/ds (% w/w) | <8.5 | 8.4 |
| Soluble protein - NSI (% w/w) | <20 | 22.8 |
| Protein content ds/ds (% w/w) | >11.5 | 14.5 |

TABLE 4
Testing results for gluten after processing.

| | Target | Measured |
|---|---|---|
| Property | Range | Property |
| Glutomatic | | |
| Gluten Index (% w/w) | >96 | 95.2 |
| Wet gluten content (155) (%) | >29.5 | 28.9 |
| Thereof dry gluten (% w/w) | >13 | 12.6 |
| Thereof protein content (% w/w) | >75 | 83.1 |
| Wet gluten content (137/1) (%) | >28 | 28.2 |
| Thereof dry gluten (% w/w) | >12 | 12.3 |
| Thereof protein content (% w/w) | >75 | 82.8 |
| Glutopeak | | |
| Peak maximum time (“PMT”) (sec.) | 70-140 | 89 |
| Maximum (“BEM”) (BE) | >40 | 53 |
| Ante maximum (“AM”) (BE) | | 22 |
| Post maximum (“PM”) (BE) | | 33 |
| Sedimentation value (mL) | >24 | 44 |
| Farinograph | | |
| Water absorption (%) | >58 | 58 |
| Dough formation (min.) | >5 | 1.9 |
| Stability (min.) | >7 | 11.5 |
| Dough softening (FE) | <70 | 36 |
| Abstehnote (—) | 3.8-4.0 | 3.8 |

TABLE 5
Testing results for starch after processing

| Property | Target Range | Measured Property |
|---|---|---|
| Starch content on ds (% w/w) | >80 | 76.9 |
| Falling number (s) | >280 | 453 |
| Amylograph | | |
| Gelatinization maximum (AE) | >500 | 2111 |
| Gelatinization temperature (° C.) | 63-68 | 92 |

Overall, the provided sample included wheat flour with middle mineral content, very high protein content, low starch content, low fiber content and good storage characteristics. The gluten developed normally, the expected gluten consistence became viscous, while vitality and elasticity were expected in average.

The washing efforts for gluten produced advantageous results, such as with starch recovery. Dough preparation was be supported by increased maturation time and temperature. The expected separation results were average gluten yield with high purity and average starch quality. With convenient dough preparation, extensive efforts within the washing stages were avoidable.

The wheat flour was also tested for particle size through sieve analysis, and total starch content was analyzed for particle size. Both sieve analysis and particle size distribution of total starch content are summarized below in Table 6.

TABLE 6
Particle size testing of flour.

| Particle size (μm) | Target Range | Measured Property |
|---|---|---|
| Flour | | |
| >250 | <2 | 0.1 |
| 125-250 | <35 | 6.3 |
| 63-125 | <35 | 53.5 |
| <63 | | 40.1 |
| A-Starch | | |
| <10 | | 13.17 |
| 10-15 | | 18.43 |
| >15 | | 68.40 |
| B-Starch | | |
| <10 | | 31.32 |
| 10-15 | | 8.37 |
| >15 | | 60.31 |
| Total Starch | | |
| <10 | <30 | 20.86 |
| 10-15 | | 13.01 |
| >15 | >55 | 66.13 |

Overall, the samples provided were composed of wheat flour characterized by a moderate mineral content, exceptionally high protein levels, low starch and fiber contents, and favorable storage properties. The gluten developed normally, though it tended to become viscous. Both vitality and elasticity were observed to be average.

The process required effort in washing the gluten to recover starch. The dough preparation benefited from extended maturation times and higher temperatures. The outcomes included an average yield of gluten with high purity and starch of average quality. With proper dough preparation, it was possible to minimize extensive efforts during the washing stages.

Thus, the proposed methods and systems used herein, which can also be used to recycle water, were effective in producing gluten and starch from wheat flour as desired.

### Example 3. Process Water from Starch

An example processing method based on FIG. 1 and FIG. 2 above was conducted including the use of different water samples in an effort to test water recycling within such systems. This laboratory test aimed to assess whether the process water produced by the operator led to significant losses in gluten and/or starch quality. For this test, several different water samples were submitted to evaluate their suitability as substitutes for tap water. The testing of the samples was the same as the methods summarized in Table 2 above.

The flour, gluten, and starch from Example 2 were further tested in stirring tests with the water samples, the results of which are summarized below in Table 7:

TABLE 7
Water testing.

| | | | Water | Water |
|---|---|---|---|---|
| Parameter | Fraction | Gluten | Sample 1 | Sample 2 |
| Yield - dry | Pentosan | 4.82 | 6.42 | 5.05 |
| mass percentage | B-starch | 3.0 | 5.5 | 4.7 |
| rate (% ds/ds) | supernatant | | | |
| | B-starch | 12.8 | 12.6 | 12.0 |
| | A-(minus)- | 7.3 | 6.8 | 6.11 |
| | starch | | | |
| | Fibers | 0.73 | 0.72 | 1.28 |
| | Gluten | 13.6 | 11.2 | 1.11 |
| | A-Starch | 57.7 | 56.7 | 59.7 |
| Protein content | Pentosan | 23.1 | 18.2 | 19.6 |
| in dry substance | B-starch | 20.6 | 19.8 | 21.1 |
| (% w/w) | supernatant | | | |
| | B-starch | 4.77 | 3.75 | 4.69 |
| | A-(minus)- | 2.65 | 1.06 | 1.00 |
| | starch | | | |
| | Fibers | 30.3 | 20.0 | 23.0 |
| | Gluten | 82.1 | 89.7 | 89.8 |
| | A-Starch | 1.96 | 0.66 | 0.58 |

Water Sample 1 included a stirring test with 50% FILTERED WATER F1 and 50% tap city water, and washing with 80% tap city water and 20% tap water from Vilsbiburg, Germany. Water Sample 2 included stirring test with 50% FILTERED WATER F2 and 50% tap city water, and washing with 80% tap city water and 20% tap water from Vilsbiburg, Germany. The Gluten column shows the whole process results from Example 2 above for comparison.

It should be noted that the production of gluten with recycled water, whether Water Sample 1 or Water Sample 2, is up past 89% w/w recovery compared to 82% w/w recovery as shown in example 1 without use of the recycled water.

The results obtained do not show significant changes during the stirring test, which includes manual separation of the phases after the spin test, manual formation and washing of the gluten content, washing of the starch fractions, and fiber removal. Depending on the anticipated quality of the flour in the future, adjustments to the mixing ratio may be necessary. Generally, the city (process) water provided can be used as a partial substitute for tap water in the wet starch process.

### ADDITIONAL EXAMPLES AND ILLUSTRATIONS

In some aspects, the techniques described herein relate to a method of processing an agricultural commodity, the method including: separating a process stream in a production facility with a mechanical separation device into a liquid stream and a solids stream, wherein the process stream includes water and the agricultural commodity; clarifying a portion of the liquid stream with a filtration system to recover water from the liquid stream; isolating protein from the liquids stream; isolating starch from the solids stream; and conveying the recovered water to the process stream in the first production facility or to a second production facility.

In some aspects, the techniques described herein relate to a method, wherein the separating includes classifying based on a pore dimension.

In some aspects, the techniques described herein relate to a method, wherein clarifying includes shunting a retentate from the solids stream to another location.

In some aspects, the techniques described herein relate to a method, wherein clarifying a portion of the liquid stream includes using a size exclusion device.

In some aspects, the techniques described herein relate to a method, wherein clarifying a portion of the liquid stream includes using a cross-flow filtration device.

In some aspects, the techniques described herein relate to a method, wherein recycling water from the liquid stream includes using the water in the slurry.

In some aspects, the techniques described herein relate to a method, wherein recycling water from the liquid stream includes using the water in a different facility.

In some aspects, the techniques described herein relate to a method, wherein recycling water from the liquid stream includes using the water in an ethanol plant.

In some aspects, the techniques described herein relate to a method, further including receiving the process stream.

In some aspects, the techniques described herein relate to a method, further including producing ethanol with the starch.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 2%, 2%, 3%, and 4%) and the sub-ranges (e.g., 0.1% to 0.5%, 2.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 20%, within 5%, or within 2% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The terms and expressions that have been employed are used as terms of description and not of limitation, and there is no intention in the use of such terms and expressions of excluding any equivalents of the features shown and described or portions thereof, but it is recognized that various modifications are possible within the scope of the examples of the present disclosure. Thus, it should be understood that although the present disclosure has been specifically disclosed by specific examples and optional features, modification and variation of the concepts herein disclosed may be resorted to by those of ordinary skill in the art, and that such modifications and variations are considered to be within the scope of examples of the present disclosure.

What is claimed is:

1. A method of processing an agricultural commodity, the method comprising:
separating a process stream in a first production facility with a mechanical separation device into a liquid stream and a solids stream, wherein the process stream includes water and the agricultural commodity;
clarifying a portion of the liquid stream with a filtration system to recover water from the liquid stream;
isolating protein from the liquids stream;
isolating starch from the solids stream; and
conveying the recovered water to the process stream in the first production facility or to a second production facility.

2. The method of claim 1, wherein the separating includes classifying based on a pore dimension.

3. The method of claim 1, wherein clarifying includes shunting a retentate from the solids stream to another location.

4. The method of claim 1, wherein clarifying a portion of the liquid stream comprises using a size exclusion device.

5. The method of claim 1, wherein clarifying a portion of the liquid stream comprises using a cross-flow filtration device.

6. The method of claim 1, wherein conveying the recovered water comprises using water in the process stream.

7. The method of claim 1, wherein conveying the recovered water comprises using the water in a different facility.

8. The method of claim 1, wherein conveying the recovered water comprises using the water in an ethanol plant.

9. The method of claim 1, further comprising receiving the process stream.

10. The method of claim 1, further comprising producing ethanol with the starch.

11. A system comprising:
a separation device configured to separate a slurry including an agricultural commodity into a first solids stream and a first liquid stream;
a feed tank coupled to receive the first liquid stream and, following a residence time, configured to provide a second solids stream; and
a filter configured to receive a second solids stream and yield a filtrate and a retentate.

12. The system of claim 11, wherein the filtrate comprises a third solids stream.

13. The system of claim 11, wherein the filtrate comprises recovered water.

14. The system of claim 11, wherein the retentate comprises one or more suspended solids.

15. The system of claim 11, wherein the filtrate comprises a permeate stream.

16. The system of claim 11, further comprising a retentate tank for receiving the first solids stream and the second solids stream.

17. The system of claim 16, wherein the retentate tank is actuatable for producing an ethanol slurry blend from the first solids stream and the second solids stream.

18. The system of claim 11, wherein the filtration system comprises a sedicanter, a decanter centrifuge, a disk stack centrifuge, a cyclone, a hydrocyclone, a settling tank, or combinations thereof.

19. The system of claim 11, wherein the separation device comprises a membrane filtration device.

20. The system of claim 11, wherein the separation device comprises multiple filtration devices.
